SEQUENCE LISTING (80 sequences)

SEQ ID NO: 1 - protein, 4 aa, synthetic construct
  REGION 1..4: note = Synthetic
WQHE

SEQ ID NO: 2 - protein, 4 aa, synthetic construct
  REGION 1..4: note = Synthetic
KWKK

SEQ ID NO: 3 - protein, 4 aa, synthetic construct
  REGION 1..4: note = Synthetic
NFKD

SEQ ID NO: 4 - protein, 4 aa, synthetic construct
  REGION 1..4: note = Synthetic
RADH

SEQ ID NO: 5 - protein, 4 aa, synthetic construct
  REGION 1..4: note = Synthetic
CSEI

SEQ ID NO: 6 - protein, 4 aa, synthetic construct
  REGION 1..4: note = Synthetic
IIHK

SEQ ID NO: 7 - protein, 4 aa, synthetic construct
  REGION 1..4: note = Synthetic
LWEY

SEQ ID NO: 8 - protein, 4 aa, synthetic construct
  REGION 1..4: note = Synthetic
IWDH

SEQ ID NO: 9 - protein, 4 aa, synthetic construct
  REGION 1..4: note = Synthetic
EPPA

SEQ ID NO: 10 - protein, 4 aa, synthetic construct
  REGION 1..4: note = Synthetic
ACDN

SEQ ID NO: 11 - protein, 5 aa, synthetic construct
  REGION 1..5: note = Synthetic
FYPAK

SEQ ID NO: 12 - protein, 4 aa, synthetic construct
  REGION 1..4: note = Synthetic
WFYP

SEQ ID NO: 13 - protein, 4 aa, synthetic construct
  REGION 1..4: note = Synthetic
YNKK

SEQ ID NO: 14 - protein, 5 aa, synthetic construct
  REGION 1..5: note = Synthetic
KALAI

SEQ ID NO: 15 - protein, 4 aa, synthetic construct
  REGION 1..4: note = Synthetic
YGCY

SEQ ID NO: 16 - protein, 5 aa, synthetic construct
  REGION 1..5: note = Synthetic
CCYKK

SEQ ID NO: 17 - protein, 5 aa, synthetic construct
  REGION 1..5: note = Synthetic
WFYPA

SEQ ID NO: 18 - protein, 4 aa, synthetic construct
  REGION 1..4: note = Synthetic
GKVF

SEQ ID NO: 19 - protein, 5 aa, synthetic construct
  REGION 1..5: note = Synthetic
NKYWF

SEQ ID NO: 20 - protein, 4 aa, synthetic construct
  REGION 1..4: note = Synthetic
KFFC

SEQ ID NO: 21 - protein, 5 aa, synthetic construct
  REGION 1..5: note = Synthetic
KYWFY

SEQ ID NO: 22 - protein, 4 aa, synthetic construct
  REGION 1..4: note = Synthetic
IPSP

SEQ ID NO: 23 - protein, 4 aa, synthetic construct
  REGION 1..4: note = Synthetic
KVFL

SEQ ID NO: 24 - protein, 5 aa, synthetic construct
  REGION 1..5: note = Synthetic
YWFYP

SEQ ID NO: 25 - protein, 5 aa, synthetic construct
  REGION 1..5: note = Synthetic
WKTYW

SEQ ID NO: 26 - protein, 4 aa, synthetic construct
  REGION 1..4: note = Synthetic
NRNY

SEQ ID NO: 27 - protein, 4 aa, synthetic construct
  REGION 1..4: note = Synthetic
FANL

SEQ ID NO: 28 - protein, 5 aa, synthetic construct
  REGION 1..5: note = Synthetic
FCKKP

SEQ ID NO: 29 - protein, 4 aa, synthetic construct
  REGION 1..4: note = Synthetic
GKIP

SEQ ID NO: 30 - protein, 4 aa, synthetic construct
  REGION 1..4: note = Synthetic
YLQK

SEQ ID NO: 31 - protein, 4 aa, synthetic construct
  REGION 1..4: note = Synthetic
WFYG

SEQ ID NO: 32 - protein, 4 aa, synthetic construct
  REGION 1..4: note = Synthetic
CYKK

SEQ ID NO: 33 - protein, 4 aa, synthetic construct
  REGION 1..4: note = Synthetic
YSSY

SEQ ID NO: 34 - protein, 4 aa, synthetic construct
  REGION 1..4: note = Synthetic
FYPA

SEQ ID NO: 35 - protein, 4 aa, synthetic construct
  REGION 1..4: note = Synthetic
TYNK

SEQ ID NO: 36 - protein, 4 aa, synthetic construct
  REGION 1..4: note = Synthetic
KKLT

SEQ ID NO: 37 - protein, 4 aa, synthetic construct
  REGION 1..4: note = Synthetic
CKKP

SEQ ID NO: 38 - protein, 4 aa, synthetic construct
  REGION 1..4: note = Synthetic
FFCK

SEQ ID NO: 39 - protein, 4 aa, synthetic construct
  REGION 1..4: note = Synthetic
YGAK

SEQ ID NO: 40 - protein, 4 aa, synthetic construct
  REGION 1..4: note = Synthetic
KNNY

SEQ ID NO: 41 - protein, 4 aa, synthetic construct
  REGION 1..4: note = Synthetic
KALA

SEQ ID NO: 42 - protein, 5 aa, synthetic construct
  REGION 1..5: note = Synthetic
KFFCK

SEQ ID NO: 43 - protein, 4 aa, synthetic construct
  REGION 1..4: note = Synthetic
RNYL

SEQ ID NO: 44 - protein, 4 aa, synthetic construct
  REGION 1..4: note = Synthetic
YLKP

SEQ ID NO: 45 - protein, 4 aa, synthetic construct
  REGION 1..4: note = Synthetic
QLGK

SEQ ID NO: 46 - protein, 4 aa, synthetic construct
  REGION 1..4: note = Synthetic
LKPF

SEQ ID NO: 47 - protein, 4 aa, synthetic construct
  REGION 1..4: note = Synthetic
KNYK

SEQ ID NO: 48 - protein, 4 aa, synthetic construct
  REGION 1..4: note = Synthetic
WKKK

SEQ ID NO: 49 - protein, 4 aa, synthetic construct
  REGION 1..4: note = Synthetic
KYWL

SEQ ID NO: 50 - protein, 4 aa, synthetic construct
  REGION 1..4: note = Synthetic
KKWK

SEQ ID NO: 51 - protein, 4 aa, synthetic construct
  REGION 1..4: note = Synthetic
KYWF

SEQ ID NO: 52 - protein, 4 aa, synthetic construct
  REGION 1..4: note = Synthetic
YPAK

SEQ ID NO: 53 - protein, 4 aa, synthetic construct
  REGION 1..4: note = Synthetic
NKKY

SEQ ID NO: 54 - protein, 4 aa, synthetic construct
  REGION 1..4: note = Synthetic
KYKI

SEQ ID NO: 55 - protein, 4 aa, synthetic construct
  REGION 1..4: note = Synthetic
NYKI

SEQ ID NO: 56 - protein, 4 aa, synthetic construct
  REGION 1..4: note = Synthetic
FCKK

SEQ ID NO: 57 - protein, 4 aa, synthetic construct
  REGION 1..4: note = Synthetic
KKKW

SEQ ID NO: 58 - protein, 4 aa, synthetic construct
  REGION 1..4: note = Synthetic
YWFY

SEQ ID NO: 59 - protein, 4 aa, synthetic construct
  REGION 1..4: note = Synthetic
KKYK

SEQ ID NO: 60 - protein, 4 aa, synthetic construct
  REGION 1..4: note = Synthetic
KFKK

SEQ ID NO: 61 - protein, 4 aa, synthetic construct
  REGION 1..4: note = Synthetic
KKKY

SEQ ID NO: 62 - protein, 4 aa, synthetic construct
  REGION 1..4: note = Synthetic
KYKK

SEQ ID NO: 63 - protein, 4 aa, synthetic construct
  REGION 1..4: note = Synthetic
YKKK

SEQ ID NO: 64 - protein, 4 aa, synthetic construct
  REGION 1..4: note = Synthetic
FKKK

SEQ ID NO: 65 - protein, 4 aa, synthetic construct
  REGION 1..4: note = Synthetic
KKKI

SEQ ID NO: 66 - protein, 4 aa, synthetic construct
  REGION 1..4: note = Synthetic
KKFK

SEQ ID NO: 67 - protein, 4 aa, synthetic construct
  REGION 1..4: note = Synthetic
KKIK

SEQ ID NO: 68 - protein, 4 aa, synthetic construct
  REGION 1..4: note = Synthetic
KKKF

SEQ ID NO: 69 - protein, 4 aa, synthetic construct
  REGION 1..4: note = Synthetic
KKLK

SEQ ID NO: 70 - protein, 4 aa, synthetic construct
  REGION 1..4: note = Synthetic
KLKK

SEQ ID NO: 71 - protein, 4 aa, synthetic construct
  REGION 1..4: note = Synthetic
KKKS

SEQ ID NO: 72 - protein, 4 aa, synthetic construct
  REGION 1..4: note = Synthetic
LKKK

SEQ ID NO: 73 - protein, 4 aa, synthetic construct
  REGION 1..4: note = Synthetic
IKKK

SEQ ID NO: 74 - protein, 4 aa, synthetic construct
  REGION 1..4: note = Synthetic
KIKK

SEQ ID NO: 75 - protein, 4 aa, synthetic construct
  REGION 1..4: note = Synthetic
KSKK

SEQ ID NO: 76 - protein, 4 aa, synthetic construct
  REGION 1..4: note = Synthetic
KKSK

SEQ ID NO: 77 - protein, 4 aa, synthetic construct
  REGION 1..4: note = Synthetic
KKPK

SEQ ID NO: 78 - protein, 4 aa, synthetic construct
  REGION 1..4: note = Synthetic
KKKL

SEQ ID NO: 79 - protein, 4 aa, synthetic construct
  REGION 1..4: note = Synthetic
KPKK

SEQ ID NO: 80 - protein, 4 aa, synthetic construct
  REGION 1..4: note = Synthetic
PKKK

(19) United States
(12) Patent Application Publication
(10) Pub. No.: US 2026/0256671 A1
(43) Pub. Date: Sep. 3, 2026

(54) MODIFIED PEPTIDES, COMPOSITION, METHOD FOR INHIBITING CONTRACTION OF MUSCLE CELLS, METHOD FOR IMPROVING THE SKIN AND USE OF A MODIFIED PEPTIDE

(71) Applicant: L'OREAL, Paris (FR)

(72) Inventors: Rodrigo DE VECCHI, Rio de Janeiro (BR); Charbel BOUEZ, Clark, NJ (US); Guillaume LEREAUX, Aulnay-sous-Bois (FR); Janet WANGARI-OLIVERO, Clark, NJ (US)

(73) Assignee: L'OREAL, Paris (FR)

(21) Appl. No.: 18/993,357

(22) PCT Filed: Jul. 13, 2022
(86) PCT No.: PCT/BR2022/050260

§ 371 Date: Jan. 10, 2025

(51) Int. Cl. A61K 8/64; A61Q 19/08; C07K 5/10

(52) U.S. Cl. CPC A61K 8/64; A61Q 19/08; C07K 5/10

(57) ABSTRACT

The invention relates to modified peptides comprising an amino acid sequence of SEQ ID NO: 1 to 80 and a N- and/or C-terminal modification, preferably with an acetyl and/or a carboxyl group modification. The present invention also relates to a composition comprising said modified peptides, to methods for inhibiting contraction of muscle cells and for improving the skin, and the use of said modified peptide.

### FIELD OF THE INVENTION

The invention relates to modified peptides comprising modifications in the N- and/or C-terminal region. The present invention also relates to a composition comprising said modified peptides, to methods for inhibiting contraction of muscle cells and for improving the skin, and the use of said modified peptide.

### BACKGROUND OF THE INVENTION

Peptides derived from natural peptide precursors isolated from, for example, snake venom, are widely used for various therapeutic or cosmetic purposes. A well-known example is captopril, whose natural peptide precursor is isolated from Bothrops jararaca snake venom. Captopril is a peptide-based drug that inhibits the angiotensin-converting enzyme, producing an antihypertensive effect. Other useful peptides prepared from natural peptide precursors include natriuretic peptides, bradykinin-potentiating peptides and sarafotoxins. Low mass proteins such as crotamine, disintegrins and three-Finger toxins are derived from snake venoms.

Some commercial peptide products contain synthetic peptides derived from natural peptides isolated from snake venom. For example, SYN®-AKE (DSM) is an effective wrinkle-smoothing compound based on a synthetic tripeptide, i.e., dipeptide diaminobutyroyl benzylamide diacetate, that mimics the activity of Waglerin 1, a polypeptide found in the venom of the Temple Viper, Tropidolaemus wagleri. SYN®-AKE acts at the postsynaptic membrane and is a reversible antagonist of the muscular nicotinic acetylcholine receptor (mnAChR). Upon binding to the mnAChR by SYN®-AKE, Na⁺ uptake is blocked at the postsynaptic membrane and muscle cell contraction is attenuated. SYN®-AKE is able to reduce signal transmission between nerves and works in a manner similar to Botox to relax muscles.

Although there are known peptides already used in the cosmetic, dermo cosmetic and dermatological areas, they have been shown to be less stable than desired, in terms of their chemical stability in a composition and also in terms of their metabolic/biologic stability after their application on a subject.

One of the main causes of the poor metabolic/biologic stability of peptides is that they are susceptible to enzymatic degradation by peptidases, which are proteases capable of cleaving, and thereby often inactivating, small peptides. Such enzymes are widely distributed on the surface of many different cell types and given the broad distribution of them in the body, several tissues including the skin have the capacity to metabolize peptides into amino acid residues.

There remains a need for bioactive peptides that have improved stability, particularly chemical and/or metabolic/biologic stability.

In view of the above, the objective of the present invention is to provide peptides that have improved metabolic/biologic and/or chemical stability in the composition and after application on a subject, particularly onto the skin.

Thus, the inventors have succeeded to overcome the problem of the state of the art and surprisingly developed new peptides comprising C- and/or N-terminal modifications that have the desired improved, said modifications preferably being with acetyl and/or carboxyl groups.

### SUMMARY OF THE INVENTION

The present invention relates to modified peptides comprising modifications in the N- and/or C-terminal region, preferably with an acetyl and/or carboxyl modification.

The present invention also relates to a composition comprising said modified peptides, to methods for inhibiting contraction of muscle cells and for improving the skin, and the use of said modified peptide in the cosmetic, dermo cosmetic and dermatologic fields.

### BRIEF DESCRIPTION OF THE DRAWINGS

FIG. 1 shows the mass spectrum of non-acetylated peptide of SEQ ID NO: 2.

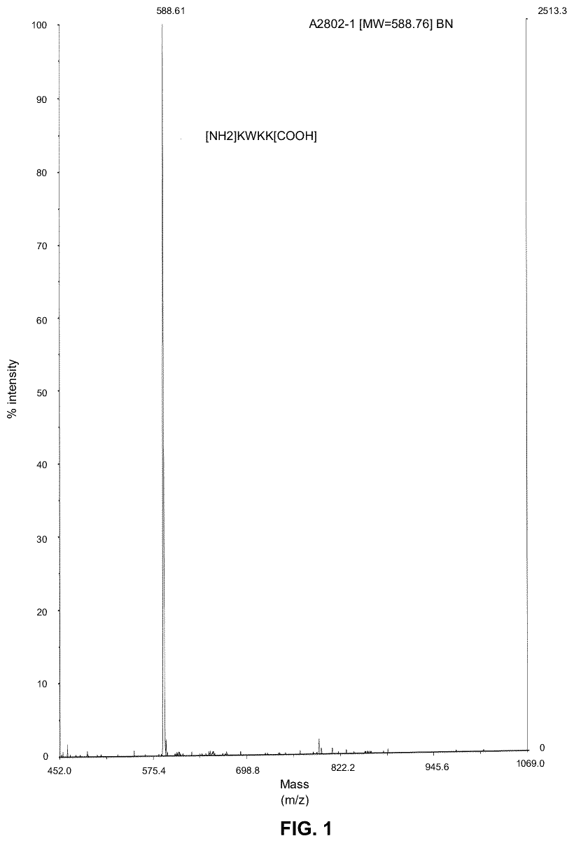

FIG. 2 shows the chromatogram and mass spectra of acetylated peptides of SEQ ID NOs: 2, 4 and 48 quantified via LC/MS of High Resolution.

FIGS. 3A and 3B show the results of a comparative skin metabolism study, wherein FIG. 3A represent the remaining percentage of non-acetylated peptide of SEQ ID NO: 2 over time and FIG. 3B represent the remaining percentage of acetylated peptide of SEQ ID NO: 2 over time in a 2-hours experiment. Doted dots represent the controls and solid dots represent the data points with metabolic activity (Skin S9).

### DETAILED DESCRIPTION OF THE INVENTION

The present invention relates to modified peptides comprising an amino acid sequence of SEQ ID NO: 1 to 80, with a N- and/or C-terminal modification. In a preferred embodiment, the N- and/or C-terminal modification is selected from acetyl and/or carboxyl group. In a preferred embodiment, the modified peptide comprises an amino acid sequence selected from SEQ ID NOs: 1 to 10, 48 and 51, with a N- and/or C-terminal modification. In more preferred embodiment, the modified peptide comprises an amino acid sequence of SEQ ID NO: 2 with a N- and/or C-terminal modification.

The term “peptide” used herein refers to a compound having two or more amino acids linked in a chain by peptide bounds, with or without a branched chain. Any amino acid in the peptide may have one or more post-translation modifications.

In a preferred embodiment, the modified peptide is a low mass peptide having no more than 5, 10, 50 or 100 amino acids. In a further preferred embodiment, the modified peptide is a tetra-peptide having four amino acids linked in a straight chain without a branched chain comprising a terminal modification.

The modified peptides of the present invention are particularly suitable for improving the skin and may be applied topically and/or associated with some medical/aesthetic procedures/devices (such as laser, radiofrequency, microneedling, among other techniques). By “improving the skin”, it may be understood one or more beneficial features such as improving elasticity, skin quality (acne scars, pores, inflammation) and firming, the appearance of pores, as well as prevent acne scars and improving the appearance of such scars, improve/prevent the appearance and aspect of wrinkles, among others.

The modified peptide may be used in the cosmetic, dermo cosmetic and/or dermatologic fields, such as a ready-to-use product to be applied by the final consumer, or a product to be applied by a health professional such as a dermatologist or a beautician for aesthetic purposes.

The present invention also relates to a composition comprising an effective amount of said modified peptide and a cosmetically, dermo cosmetically, and/or dermatologically acceptable vehicle, diluent, or carrier.

The composition may comprise the modified peptide at a concentration of about 0.01-500 μM, about 0.1-100 μM, about 1-50 μM or about 1-10 μM, for example, 1 or 10 μM.

The composition may be a cosmetic composition and further comprise a cosmetically acceptable vehicle, diluent, or carrier. The composition may be a dermo cosmetic composition and further comprise a dermo cosmetically acceptable vehicle, diluent, or carrier. The composition may be a dermatologic composition and further comprise a dermatologically acceptable vehicle, diluent, or carrier. Cosmetically, dermo cosmetically, and dermatologically acceptable vehicle, diluent, or carrier are known from the art and may be selected by the judgement of a technician.

The composition may be in the form of a lotion, gel, milk, serum, cream, emulsion, liquid, lyophilized powder, aerosol, or spray. The composition may be incorporated in microneedle systems, such as hydrogel patches or devices. The composition may further contain other compounds usually used in the field, such as surfactants, thickeners or gelling agents, preservatives, agents basifying or acidifying agents well known in the art, and in amounts sufficient to obtain the desired form of presentation, in particular of lotion more or less thickened, gel, emulsion, or cream. The composition may be in a pressurized aerosol or spray form from a pump bottle.

The term “an effective amount” refers to an amount of a modified peptide or a composition comprising the modified peptide according to this invention that is required to achieve a stated goal. The effective amount may vary depending on the nature of the modified peptide or of the composition, the type of the target cells, treatment time, and the stated goal. A specific effective amount for a given peptide or a given composition may generally be set by the judgement of a technician.

The present invention also relates to a method for inhibiting contraction of muscle cells, wherein such cells are contacted with an effective amount of the modified peptide or the composition comprising thereof, said contact resulting in the inhibition of contraction of the muscle cells. The inhibition may be irreversible.

According to the inhibition method, the muscle cells may be treated for at least about 0.5, 1, 2, 5, 10, 12, 18, 24 or 48 hours, for example, at least about 5 or 24 hours. The contraction of the muscle cells may be inhibited by at least about 5%, 10%, 20%, 30%, 40%, 50%, 60%, 70%, 80%, 90%, 95% or 99% as compared with that before the treatment.

In some embodiments, the muscle cells are in a muscle of a subject. Upon treatment of the muscle cells with the modified peptide or the composition comprising thereof, the muscle may be relaxed.

The invention also relates to a method for improving the skin, comprising applying an effective amount of the modified peptide or the composition comprising thereof. The skin may be treated at least once, twice or three times daily and/or for at least about 7, 14, 21, 28, 60 or 90 days.

The present invention also relates to the use of the modified peptide for manufacturing a composition or a product for cosmetic, dermo cosmetic and/or dermatologic treatments. In a preferred embodiment, said use is through the aid of an aesthetic device for laser, radiofrequency and/or for microneedling.

A person skilled in the art may easily determine which field of application is of interest based on the type of composition that is desired, for example, a treatment based on a ready-to-use cream, of a dermatologic composition to be applied by a health care professional onto the consumer.

### Terms

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

Although the invention is illustrated and described herein with reference to specific embodiments, the invention is not intended to be limited to the details shown. Rather, various modifications may be made in the details within the scope and range of equivalents of the claims without departing from the invention.

### Peptide Modifications

As already mentioned, the term “peptide” used herein refers to a compound having two or more amino acids linked in a chain by peptide bounds, with or without a branched chain. Any amino acid in the peptide may have one or more post-translation modifications.

In a peptide, the carboxyl group of one amino acid is bonded to the amino group of another. One end of the peptide has a free amino group, while the other end has a free carboxyl group, forming the amino (N-terminus—[NH₂]) and carboxyl (C-terminus—[COOH]) ends, respectively. In one embodiment, the modified peptide is a tetra-peptide having four amino acids linked in a straight chain without a branched chain comprising a terminal modification.

The “terminal modification” can occur on the N- and/or C-terminal end of the modified peptide and may include, but are not limited to, amino and carboxy terminals modifications, substitutions and/or conjugations. Terminal modifications may occur with an acetyl group, a methyl group, a phosphate group, a carboxyl group, a fatty acid including capryloyl, capryl and lauryl groups, an amide, an N-alkyl amide, an aldehyde, a urea, an alkylamine etc. In a preferred embodiment, terminal modification comprises the modification of the N- and/or C-terminal end with an acetyl and/or carboxyl group. In one preferred embodiment, the peptide is N-acetylated. In another preferred embodiment, the peptide is carboxylated.

Acetylation describes a reaction wherein the positive charge on the N-terminal of peptides is removed. Carboxylation describes a reaction wherein a carboxyl group is incorporated in the terminal end of the peptide. Amidation describes a reaction wherein the C-terminal end of the peptide is synthesized as an amide to neutralize the negative charge of the C-terminal COOH.

Terminal modifications can be synthetized via usual techniques in the field. The modified peptide may be chemically and/or enzymatically synthetized and, in a preferred embodiment, the peptide is enzymatically synthetized. Suitable enzymes may include transferases, which are enzymes that transfer a group from one compound to another and are classified according to their donor: acceptor group scheme. In another preferred embodiment, the peptide is chemically synthetized.

Terminal modifications can increase the modified peptide cosmetic, metabolic/biologic and/or chemical stability by preventing the N- and/or C-terminal degradation by peptidases and/or by modifying the physicochemical properties of the peptides which may include peptide length, molecular mass, hydrophobicity etc.

### EXAMPLES

### Example 1—Modified Peptide

The modified peptide of the present invention comprises an amino acid sequence selected from SEQ ID NOs: 1 to 80, as shown in Table 1.

TABLE 1
Peptide Sequences

| SEQ | | SEQ | | SEQ | | SEQ | |
|---|---|---|---|---|---|---|---|
| ID NO | Peptide | ID NO | Peptide | ID NO | Peptide | ID NO | Peptide |
| 1 | WQHE | 21 | KYWFY | 41 | KALA | 61 | KKKY |
| 2 | KWKK | 22 | IPSP | 42 | KFFCK | 62 | KYKK |
| 3 | NFKD | 23 | KVFL | 43 | RNYL | 63 | YKKK |
| 4 | RADH | 24 | YWFYP | 44 | YLKP | 64 | FKKK |
| 5 | CSEI | 25 | WKTYW | 45 | QLGK | 65 | KKKI |
| 6 | IHK | 26 | NRNY | 46 | LKPF | 66 | KKFK |
| 7 | LWEY | 27 | FANL | 47 | KNYK | 67 | KKIK |
| 8 | IWDH | 28 | FCKKP | 48 | WKKK | 68 | KKKF |
| 9 | EPPA | 29 | GKIP | 49 | KYWL | 69 | KKLK |
| 10 | ACDN | 30 | YLQK | 50 | KKWK | 70 | KLKK |
| 11 | FYPAK | 31 | WFYG | 51 | KYWF | 71 | KKKS |
| 12 | WFYP | 32 | CYKK | 52 | YPAK | 72 | LKKK |
| 13 | YNKK | 33 | YSSY | 53 | NKKY | 73 | IKKK |
| 14 | KALAI | 34 | FYPA | 54 | KYKI | 74 | KIKK |
| 15 | YGCY | 35 | TYNK | 55 | NYKI | 75 | KSKK |
| 16 | CCYKK | 36 | KKLT | 56 | FCKK | 76 | KKSK |
| 17 | WFYPA | 37 | CKKP | 57 | KKKW | 77 | KKPK |
| 18 | GKVF | 38 | FFCK | 58 | YWFY | 78 | KKKL |
| 19 | NKYWF | 39 | YGAK | 59 | KKYK | 79 | KPKK |
| 20 | KFFC | 40 | KNNY | 60 | KFKK | 80 | PKKK |

### Example 2—Peptide Quantification Via LC/MS High Resolution

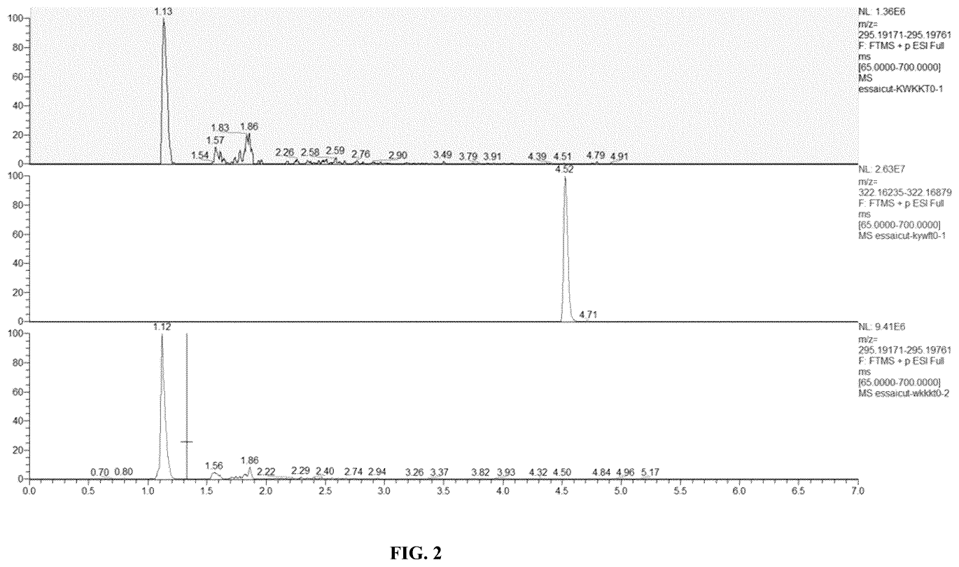

The acetylated peptides were quantified with a UPLC Vanquish (Thermo™) chromatography system coupled to a mass spectrometer Q-exactive (Thermo™). The peptides were ionized with Electrospray positive ion mode and detected with full scan mode with resolution power at 35000 amu. Mass spectra shown two-charged ions and the quantification was done on ion m/z 295.195 amu (with z=2, FIG. 2) for peptides KWKK (SEQ ID NO: 2) and WKKK (SEQ ID NO: 48) and ion m/z 322.166 (with z=2, FIG. 2) for tetrapeptide KYWF (SEQ ID NO: 51). Data was treated with Excalibur software and is depicted on FIG. 2.

### Example 3—Comparative Skin Metabolism Study

The metabolic/biologic stability of acetylated and non-acetylated peptides of SEQ ID NO: 2 was studied with subcellular S9 fractions (S9) obtained from reconstructed human skin models Episkin™, and liver. The half-life of peptides was determined quantitatively, and the qualitative appearance of potential metabolites was studied. The protocol for the metabolism study with skin S9 is showed below.

TABLE 2
Protocol for Skin Metabolism Study with Skin S9

| | Liver and Skin S9 |
|---|---|
| [Biological material] (mg prot./mL) | 2 |
| [peptides] (μM) | 5 |
| Buffer | Phosphate buffer (100 mM, |
| | pH 7.4) |
| [Cofactors] (μM): | None |
| Kinetic at 37° C.: | 0-120 min |
| Analytical method: | |
| Quantification of substrate | LC/MS |
| Identification of metabolites | LC/MS |

Control performed in the same experimental conditions without S9 was also done in order to checked if disappearance of parent compound is related of enzymatic activities. At different time points (0, 5, 10, 15, 30, 60 and 120 min), 100 μL of medium are quenched with 200 μL of methanol 0.1% formic acid. Samples were then analyzed with LC/MS to quantify peptides. Potential metabolites (loss of one or two amino acids on the C-terminal end) were identified with LC/MS.

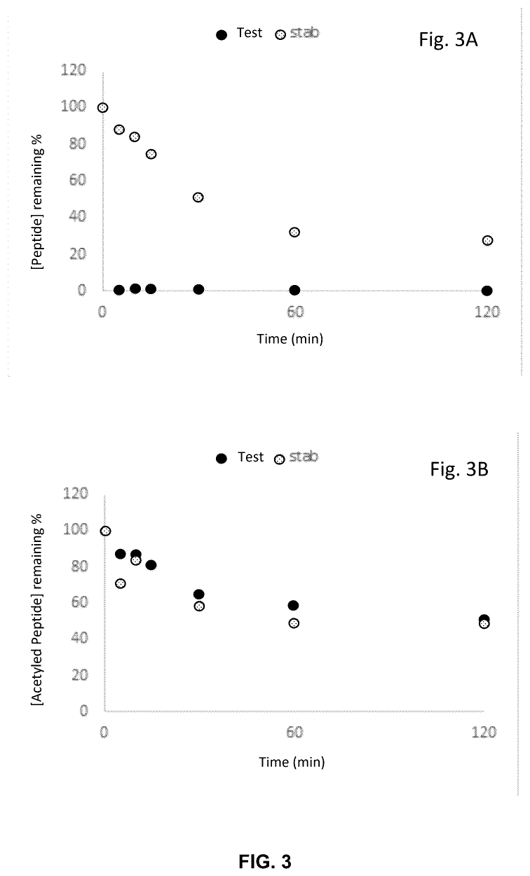

Half-life of the test peptide disappearance derives from a curve fitting through data of the residual concentration of the parent peptide versus time using GraphPad software (V. 5.02). The percentage of peptides remaining over time are depicted on FIG. 3.

It is clear from FIGS. 3A and 3B that non-acetylated peptide shows a very rapid decrease over time with a half-life below 5 min, while the acetylated peptide shows the same behavior with or without the metabolically active fraction.

1-16. (canceled)

17. A modified peptide comprising an amino acid sequence of SEQ ID NO: 1 or 2, with an N- and/or C-terminal modification.

18. The peptide of claim 17 comprising the amino acid sequence of SEQ ID NO: 1.

19. The peptide of claim 17 comprising the amino acid sequence of SEQ ID NO: 2.

20. The peptide of claim 17, wherein the N- and/or C-terminal modification is with an acetyl and/or a carboxyl group.

21. The peptide of claim 17 comprising an N-terminal modification with an acetyl and/or carboxyl group.

22. The peptide of claim 17, wherein the C-terminal modification with an acetyl and/or carboxyl group.

23. A composition comprising the modified peptide of claim 17 and a cosmetically, dermo-cosmetically, and/or dermatologically acceptable vehicle, diluent, or carrier.

24. The composition of claim 23 in the form of a lotion, gel, milk, serum, cream, emulsion, liquid, lyophilized powder, aerosol, or spray.

25. A method for inhibiting contraction of muscle cells comprising treating the muscle cells with an effective amount of the modified peptide of claim 17 and contracting the muscle cells.

26. A method for improving the appearance of skin comprising applying an effective amount of the modified peptide of claim 17 to the skin.

27. The method of claim 26, wherein application of the effective amount of the modified peptide reduces appearance of wrinkles or improves skin quality.

28. A method for manufacturing a composition for cosmetic, dermo-cosmetic, or dermatologic treatment comprising incorporating a modified peptide of claim 17 into the composition.

29. A method for treating skin comprising applying an effective amount of the modified peptide of claim 17 to the skin and improving the appearance of the skin.

30. The method of claim 29, wherein the method further comprises treating the skin with an aesthetic device, radiofrequency, microneedling, or a combination thereof.

31. The method of claim 29, wherein the method further comprises treating the skin with a laser.
